(19) United States
(12) Patent Application Publication
(10) Pub. No.: US 2010/0159024 A1
(43) Pub. Date: Jun. 24, 2010

(54) Method and apparatus for the extraction and collection of coelomic fluid of sea stars

(76) Inventors: Richard Ablett, Charlottetown (CA); Mike McKenna, Charlottetown (CA); Stephen McKenna, Dartmouth (CA); Kevin Sibley, Valley (CA)

Correspondence Address: THE NATH LAW GROUP, 112 South West Street, Alexandria, VA, 22314, US

(21) Appl. No.: 12/591,167

(22) Filed: Nov. 10, 2009

Related U.S. Application Data
(60) Provisional application No. 61/193,242, filed on Nov. 10, 2008.

(51) Int. Cl. A61K 35/56; A01K 61/00; A22C 18/00; A01N 61/00; A61P 43/00; A01P 1/00

(52) U.S. Cl. 424/537; 119/201; 452/183; 452/154

(57) ABSTRACT

A method and apparatus are provided for the extraction and collection of coelomic fluid of sea stars. The method includes feeding sea stars, one at a time, from storage into the mouth of a longitudinally-moving gripper. The method includes moving the sea stars in a downstream direction by means of the longitudinally-moving gripper. The method includes severing the tip of the arm of the sea star. The method includes allowing the coelomic fluid to drain out of the severed arm of the sea star; The method includes accumulating the drained coelomic fluid. The method includes collecting the accumulated drained coelomic fluid. Finally, the method includes discharging the sea stars from the longitudinally-moving gripper.

### BACKGROUND OF THE INVENTION

1. Field of the Invention

This invention relates to a method and apparatus for the extraction and collection of coelomic fluid of echinoderms, especially sea stars.

2. Description of the Prior Art

The coelomic fluid of many species of echinoderms, especially, sea stars, possesses valuable characteristics. Among the characteristics are those products which are bioactive agents, which repel, or are toxic to, micro-organisms, such as bacteria, yeast, and diatoms. The bioactive repellents from especially sea stars are also selective against microorganism, such as mollusks and crustaceans. Such bioactive repellents may be formulated to provide antifouling compositions.

It is also believed that the coelomic fluids of echinoderms, especially sea stars, possess sperm immotility and anti fertility characteristics.

It is also believed that the coelomic fluids of echinoderms, especially sea stars possess anti-adhesive and anti-inflammatory characteristics.

However, even though the coelomic fluids of echinoderms, especially sea stars possess such useful qualities, industrially-applicable methods for extracting and collecting such coelomic fluids are presently lacking.

Common general knowledge has suggested that the easiest and simplest method of coelomic fluid extraction from echinoderms, especially sea stars was to position the sea star vertically, excising the tip of one or more arms and allowing the fluid to gravity feed from the animal. This method, however, was both very slow, labor intensive and time consuming.

Some patents have suggested other ways of extracting and collecting coelomic fluid from echinoderms, especially sea stars. Among them are the following:

U.S. Pat. No. 6,991,810, “Product From Starfish” issued Jan. 31, 2006, assigned to King's College London, London (GB) which provided a method for extracting a product from the brittlestar Ophiocomina nigra, the cushion star Porania pulvillus and the starfish or sea star Marthasterias glacialis. The method included the steps of collection mucous from brittlestar Ophiocomina nigra, the cushion star Porania pulvillus or the starfish or sea star Marthasterias glacialis; removing particulate material by centrifugation; subjecting the supernatent to column chromatography; eluting the material from the chromatography column; and optionally dialyzing the elated material against distilled water. The product is said to be a glycoprotein having a molecular weight of about 1,100 kDa.

As taught by this patentee, the mucus was aspirated from the dorsal surface of these species using a fine glass pipette connected to a reservoir under suction. The mucus collected by aspiration was a viscous acidic liquid. Size exclusion chromatography of mucus from all three of those species of echinoderm produced the same characteristic chromatograms.

Obviously, the procedures taught by this patentee are not industrially-applicable.

U.S. Pat. No. 5,695,552 issued Dec. 9, 1997 to Taylor also provided methods or the recovery of coelomic fluid from various sea creatures including coelomic fluid extraction from echinoderms, especially sea stars.

In one procedure as taught by the patentee, the sea creatures were cleansed, and frozen until ready for further processing. The frozen sea creatures were then blended and homogenized in distilled water. The resulting homogenate was then lyophilized, resuspended in hexane and sonicated. The resulting mixture was then centrifuged resulting in a supanatant and a pellet. The solvent was then removed from the supanatant using a vortex vacuum evaporator. The pellet was then extracted with ethyl acetate and centrifuged. The resulting supernatant is separated from the resulting pellet. The pellet was further extracted with methanol and centrifuged. Solvent was removed from the resulting supanatant using a vortex vacuum evaporator tro provide the desired product.

In an alternative embodiment as taught by the patentee, the collected, cleansed creatures were blended and extracted overnight by immersion in solvent, e.g., methanol in a beaker which was shaken. After extraction, the liquid was removed and centrifuged and the supernatant was vacuum dried to obtain a solvent extract concentrate. The remains in the beaker were air dried and subjected to further solvent extraction, e.g., ethyl acetate and hexane extraction by repeating the procedures of this embodiment.

Thus, as described in this patent, polar or semi-polar solvents were used as the vehicles for obtaining active repellents from the above-identified sea creatures. Any non-polar or semi-polar solvent was suitable as long as it did not adversely react with the active repellent constituent(s) to reduce activity. Suitable solvents include ethanol, methanol, ethyl acetate, hexane, chloroform, acotonitrile and dimethylformamide.

Obviously, the procedures taught by this patentee are not industrially-applicable.

It is known that echinoderms, especially sea stars, possess a unique water vascular system, a network of fluid-filled canals that function in gas exchange, feeding, and secondarily in locomotion. The system comprises a central ring, the hydrocoel, and radial ambulacra stretching along

each limb of the organism. This system also assisted with the distribution of nutrients through the animal.

### AIMS OF THE INVENTION

One aim of the invention is to provide an industrially applicable method for the extraction and collection of coelomic fluid of sea stars

Another aim of the invention is to provide an industrially applicable apparatus for the extraction and collection of coelomic fluid of sea stars

The invention in its general form will first be described, and then its implementation in terms of specific embodiments will be detailed with reference to the drawings following hereafter. These embodiments are intended to demonstrate the principle of the invention, and the manner of its implementation. the invention in its broadest sense and more specific forms will then be further described, and defined, in each of the individual claims which conclude this specification

### SUMMARY OF THE INVENTION

### Statement of Invention

By one broad aspect, the present invention takes advantage of the above-described unique water vascular system, of sea stars to provide a method for the extraction and collection of coelomic fluid from echinoderms, especially sea stars. The method includes the step of feeding echinoderms, especially sea stars with at least one of the arms of the sea star extending vertically downward though a horizontally-moving, oppositely- rotating gripper conveyor system which moves in an endless horizontally-oriented track. The method includes the step of severing the bottom of the at least one of the arms of the sea star either just as the sea star enters the nip between the horizontally-moving, oppositely-rotating gripper conveyor system which moves in an endless horizontally-oriented track, or at any time during the passage of the sea star through the horizontally-moving, oppositely- rotating gripper conveyor system which moves in an endless horizontally-oriented track. This step exposes the water vascular system of the sea star and allows the coelomic fluid to drain out. The method includes the step of collecting that expelled coelomic fluid in a collecting vessel. The method includes the step of discharging the sea star from the horizontally moving counter rotating gripper conveyor system, into a waste container.

Another broad aspect the present invention provides an apparatus for the extraction and collection of coelomic fluid of sea stars. The apparatus includes a horizontally-movable c gripper conveyor system including a pair of closely-spaced-apart gripper belts constructed for movement in an endless track in opposite directions. The apparatus includes motor means for moving the pair of closely spaced apart gripper belts in an endless track in opposite directions. to provide a vertical gripping inlet nip between the two belts. The apparatus includes severing means downstream of the nip for severing the bottom of the at least one of the vertically downwardly extending arms of the sea star. The apparatus includes a collection trough below the a pair of closely-spaced-apart gripper belts for collecting drained coelomic fluid. a pair of closely-spaced-apart gripper belts a collection vessel in liquid communication with the collection trough.

### Other Features of the Invention

By one feature of the method aspect of the invention the method is carried out continuously

By another feature of the method aspect of the invention the method is carried out by continuously feeding the sea stars manually.

By another feature of the method aspect of the invention the method is carried out by continuously feeding the sea stars by a machine feeding system.

By another feature of the method aspect of the invention the tips of the arms of the sea star are severed immediately upon entering vertical gripping inlet nip between the two belts.

By another feature of the method aspect of the invention the tips of the arms of the sea star are severed during the passage of the sea star between the two belts.

By another feature of the method aspect of the invention the sea star are squeezed between the two belts in order to assist in the drainage of the coelomic fluid therefrom.

By another feature of the method aspect of the invention the coelomic fluid is refrigerated for storage thereof.

By one feature of the apparatus aspect of the invention the collection trough is covered by a screen.

By another feature of the apparatus aspect of the invention the bottom of the collection trough is sloped to a single drainage outlet.

By another feature of the apparatus aspect of the invention, the apparatus is part of a system for the extraction and collection of coelomic fluid of sea stars, and includes a waste container at the discharge end of the counter rotating belts conveyor to contain sea stars as they are discharged from the discharge end of the counter rotating belts conveyor.

By another feature of the apparatus aspect of the invention, the apparatus is part of a system for the extraction and collection of coelomic fluid of sea stars, and includes a filter unit for the filtration of the collected coelomic fluid of the sea stars.

By another feature of the apparatus aspect of the invention, the apparatus is part of a system for the extraction and collection of coelomic fluid of sea stars, and includes a freezer for the storage of the filtered collected coelomic fluid of the sea stars.

The foregoing summarizes the principal features of the invention and some of its optional aspects. the invention may be further understood by the description of the preferred embodiments, in conjunction with the drawings, which now follow.

### BRIEF DESCRIPTION OF THE DRAWINGS

In the accompanying drawings,

FIG. 1 is a schematic representation, in flow sheet form, of the method for the extraction and collection of coelomic fluid of sea stars;

FIG. 2 is a side elevational view of the apparatus part of the system for the extraction and collection of coelomic fluid of sea stars;

FIG. 3 is a top plan view of the apparatus part of the system for the extraction and collection of coelomic fluid of sea stars;

FIG. 4 is an end view of the inlet, nip end of the apparatus part of the system for the extraction and collection of coelomic fluid of sea stars; and

FIG. 5 is an end view of the discharge end of the apparatus part of the system for the extraction and collection of coelomic fluid of sea stars.

### DESCRIPTION OF PREFERRED EMBODIMENTS

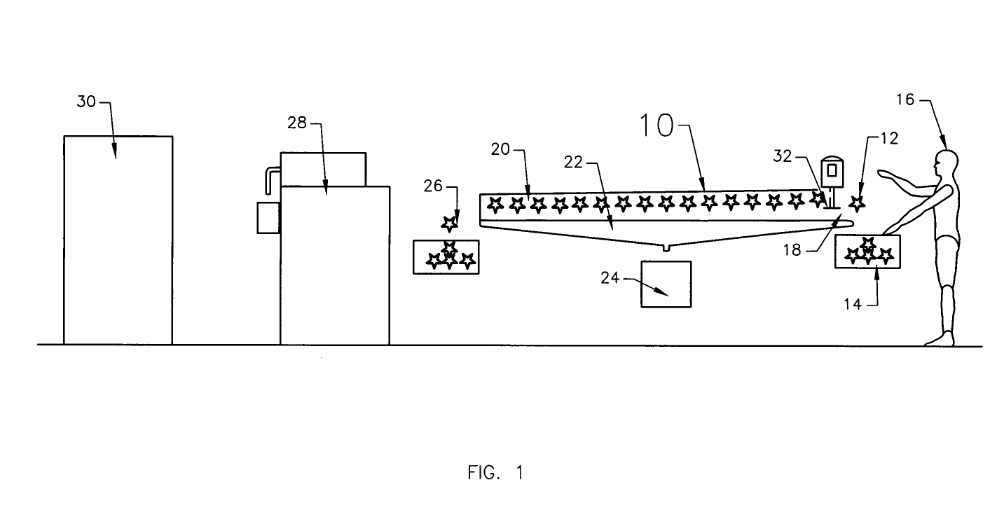

### Description of FIG. 1

The method of a broad aspect of the present invention is schematically illustrated in a flow diagram in FIG. 1. In a first step, sea stars 12 are fed, one at a time, from storage 14 by manual (human) means 16 into the mouth 18 of a longitudinally-moving gripper 20. The sea stars 12 are driven to move them in a downstream direction. The longitudinally-moving gripper 20 will be described in detail hereinafter. The sea stars 12 may, alternatively, be fed by an automated mechanical feeder (not shown). In the next step, the tip of the arm of the sea star 12 is severed e.g., by blade means 32, either at the onset of the travel of the sea star 12 along the longitudinally-moving gripper 20, or at any time as the sea star 12 is urged through the longitudinally-moving gripper 20. In the next step, the sea stars 12 are preferably squeezed during its passage through the longitudinally-moving gripper 20 to assist in the draining of the coelomic fluid from the sea stars 12. In the next step, the drained coelomic fluid is accumulated at 22 and is then collected at 24. In the next step, the sea stars 12 are discharged at 26 from the longitudinally-moving gripper 20.

In preferred next steps, the collected coelomic fluid is transferred and filtered at 28 to filter out any debris, and is then frozen at 30 until needed.

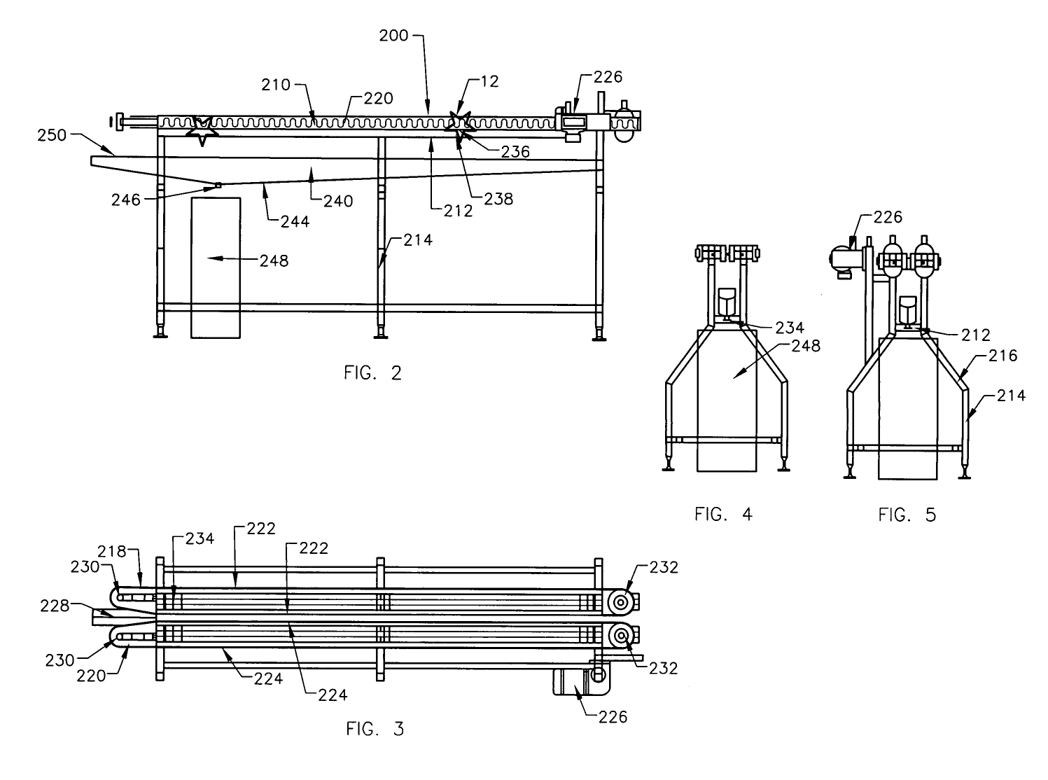

### Description of FIG. 2, FIG. 3, FIG. 4 and FIG. 5

As shown in these four FIGURES, the conveyor apparatus 210 of the system 200 for the extraction and collection of coelomic fluid of sea stars 12 includes a support table 212 provided with, preferably three, height adjustable legs 214. The legs 214 extend upwardly and then converge inwardly at 216 to provide the support table 212 for the conveyor apparatus 210. The conveyor apparatus 210 comprises two vertically-oriented, closely-spaced-apart, longitudinally movable belts 218, 220, which include compressible facings 222, 224 for gripping and preferably squeezing the sea stars 12 therebetween. The belts 218, 220 are constructed to be driven by suitable motor means 226 to travel in an endless track in opposite longitudinally-movable directions so as to provide a gripping nip or mouth 228 between the two belts 218, 220. The belts 218,220 are entrained around forward 230 and rear 232 sprockets. A cutting blade 234 is provided at the inlet, nip or mouth end 228 of the two belts 218, 220 to sever the tip 236 ends of the arm 238 of the sea star 12, and thus allow the coelomic fluid to drain. This action is preferably aided by the sea stars 12 being squeezed between the two closely-spaced-apart belts 218,220.

A trough 240 is disposed below the table 212 which supports the conveyor apparatus 210. The trough 240 includes a sloped bottom 244 to allow the coelomic fluid to accumulate and to run down to the discharge opening 246 into a collection vessel 248 which is situated below the collection trough 240. In addition, the collection trough is covered with a screen 250

In summary, the sea star coelomic fluid extraction system 200 provided herein consists of a moving gripper conveyor belt combination 218, 220 (e.g. a 3″ wide Intralox™ plastic module with rubber pads attached by plastic ties), a motor 2226 (e.g., a ¼ HP TEFC drive gear motor with a vari-frequency controller for belt speed adjustment), a graded trough/flume 240 and a cutting blade 234 (e.g. a horizontally mounted electric bandsaw) to excise one or more arms 238 of the sea star 12. The trough/flume 240 empties into a collection vessel, or small refrigerated storage cabinet 248 (e.g., one which is 20″×23″×34″ high), which is located below the trough/flume 240 is used to refrigerate the collected coelomic fluid.

The graded trough/flume, support structures and main body of the extraction system 200 is preferably fabricated from stainless steel. The extraction system 200 is supported by vertical supports 214 (e.g., three vertical supports in the form of a tripod), complete with adjustable leveling feet. In one embodiment, the dimensions of the table are approximately 14′×1′-6″×4′ high.

The operation of the sea star coelomic fluid extraction unit is as follows:

Sea stars are passed vertically oriented across a thin (e.g. about 0.6 mm thickness) cutting blade, located at one end of the unit, to excise the tip of the arm and is positioned between two moving pinch belts (gripper conveyors) until the sea star is held firmly in place. As the belt moves forward, the coelomic fluid gravity feeds from the sea star and is collected in a sloped trough/flume located along the entire length of the gripper belt. The fluid retained in the trough is directed to a refrigerated reservoir located under the trough for short term storage. A small pore (e.g., approximately 875 microns) stainless steel screening material covers the trough and retains any large particles/debris that may be present in the fluid. The gripper conveyors move at a specific speed (e.g., about 2 to about 5 feet per minute, as programmed in a variable speed motor controller) holding the sea star in a vertical position for a specific duration, e.g. about 1 to about 25 minutes, to ensure fluid removal is complete, following which the two belts separate and the sea star falls into a waste container for disposal.

The sea star coelomic fluid extraction unit is preferably designed to process ten—8″ starfish per minute with two workers. The coelomic fluid is transferred from the extraction unit for sub-micron filtering. A pump, e.g., a Masterflex™ peristaltic pump propels raw fluid from a storage container through the filters. The filtration manifold preferably consists of four filters, arranged in a series as follows: 5 micron, 1 micron, 0.5 micron and 0.2 micron. A known volume, e.g., about 10 to about 25 cc of filtered fluid is transferred to a plastic bag for vacuum sealing. The fluid filled bag is placed in the vacuum sealer and placed under negative pressure (vacuum) for about 15 to about 25 seconds and then the bag is heat sealed. Sealed bags of fluid are placed in a blast freezer for rapid freezing prior to transfer to long term (e.g., about 1 to about 3 months) refrigerated storage

### CONCLUSION

An efficient method and system and apparatus for harvesting and collecting significant volumes of sea star coelomic fluid has thus been provided herein, which enables the large number of sea stars to be processed, by a faster and more efficient method and using a novel apparatus

The foregoing has constituted a description of specific embodiments showing how the invention may be applied and put into use these embodiments are only exemplary. The invention in its broadest, and more specific aspects is further described and defined in the claims which follow.

These claims, and the language used therein are to be understood in terms of the variants of the invention which have been described. They are not to be restricted to such variants, but are to be read as covering the full scope of the invention as is implicit within the invention and the disclosure that has been provided herein.

1-12. (canceled)

13. A method for the extraction and collection of coelomic fluid of sea stars comprising:
feeding sea stars, one at a time, from storage into the mouth of a longitudinally-moving gripper;
moving the sea stars in a downstream direction by means of the longitudinally-moving gripper;
severing the tip of the arm of the sea star;
allowing the coelomic fluid to drain out of the severed arm of the sea star;
accumulating the drained coelomic fluid;
collecting the accumulated drained coelomic fluid; and
discharging the sea stars from the longitudinally-moving gripper.

14. A method for the extraction and collection of coelomic fluid of sea stars comprising:
providing a horizontally moving gripper conveyor system having endless belts moving in opposite directions along a defined track, the belts extending vertically downwardly;
feeding sea stars through the horizontally moving gripper conveyor with one arm of the sea star extending vertically downwardly below the vertical extent of the belts;
severing the bottom tip of one of the arms of the sea star which extends vertically downwardly below the vertical extent of the belts to expose the water vascular system of the sea star;
allowing the coelomic fluid of the sea star to drain;
collecting the drained coelomic fluid; and
discharging the sea stars from the horizontally moving gripper conveyor system.

15. The method of claim 14, including squeezing the sea stars by means of the horizontally moving counter rotating gripper conveyor system to assist in the drainage of the coelomic fluid from the sea stars.

16. The method of claim 13, wherein the sea stars are fed manually by manual (human) means.

17. The method of claim 14, wherein the sea stars are fed manually by manual (human) means.

18. The method of claim 13, wherein the sea stars are fed by an automated mechanical feeder.

19. The method of claim 14, wherein the sea stars are fed by an automated mechanical feeder.

20. The method of claim 13, wherein the tip of the arm of the sea star is severed at the onset of the travel of the sea star along the longitudinally-moving gripper.

21. The method of claim 13, wherein the tip of the arm of the sea star is severed at any time as the sea star is urged through the longitudinally-moving gripper.

22. The method of claim 13, wherein the sea stars are squeezed during its passage through the longitudinally-moving gripper to assist in the expelling of the coelomic fluid from the sea star.

23. An apparatus for the extraction and collection of coelomic fluid of sea stars comprising:
a horizontally movable gripper conveyor system including a pair of closely-spaced apart, vertically-oriented gripper belts;
means for moving the pair of gripper belts in an opposite longitudinal directions along a longitudinal track, to provide a vertical gripping nip between the two belts;
severing means downstream of the nip and disposed vertically below the vertical downward extent of the bottom of the belts;
a collection trough below the vertical extent of the belts for collecting drained coelomic fluid; and
a collection vessel in liquid communication with the collection trough.

24. The apparatus of claim 23, wherein the collection trough is covered by a screen.

25. The apparatus of claim 23, wherein the bottom of the collection trough is sloped to a single drainage outlet.

26. A system for the extraction and collection of coelomic fluid of sea stars comprising:
the apparatus of claim 23, including a collection unit at the discharge end of the counter rotating belts conveyor to contain sea stars as they are discharged from the discharge end of the conveyor.

27. The system of claim 26, including:
a filter unit for the filtration of the collected coelomic fluid of the sea stars.

28. The system of claim 26, including:
a freezer of the storage of the filtered collected coelomic fluid of the sea stars.
